SEQUENCE LISTING (11 sequences)

SEQ ID NO: 1 - DNA, 13 bp, Artificial Sequence
  synthesized
acagucggucgcc

SEQ ID NO: 2 - DNA, 13 bp, Artificial Sequence
  synthesized
acagtcggtcgcc

SEQ ID NO: 3 - DNA, 34 bp, Artificial Sequence
  synthesized
ggcgacccugaugaggccgaaaggccgaaacugu

SEQ ID NO: 4 - DNA, 24 bp, Artificial Sequence
  synthesized
cugangagncnnnnnngncgaaac

SEQ ID NO: 5 - DNA, 24 bp, Artificial Sequence
  synthesized
cugangagncnnnnnngncgaaan

SEQ ID NO: 6 - DNA, 25 bp, Artificial Sequence
  synthesized
cugangagnucggaaacgacgaaac

SEQ ID NO: 7 - DNA, 25 bp, Artificial Sequence
  synthesized
cugangagnucggaaacgacgaaan

SEQ ID NO: 8 - DNA, 23 bp, Artificial Sequence
  synthesized
ccuaagucgugagauauggnnnn

SEQ ID NO: 9 - DNA, 24 bp, Artificial Sequence
  synthesized
ccuaagucgugagauauggnnnnn

SEQ ID NO: 10 - DNA, 25 bp, Artificial Sequence
  synthesized
ccuaagucgugagauauggnnnnnn

SEQ ID NO: 11 - DNA, 24 bp, Artificial Sequence
  synthesized
cugaugaggccgaaaggccgaaac

(19) United States
(12) Patent Application Publication
(10) Pub. No.: US 2015/0140020 A1
(43) Pub. Date: May 21, 2015

(54) PHARMACEUTICAL COMPOSITION FOR TREATING ADVERSE REACTIONS DUE TO ADMINISTRATION OF SPIEGELMERS

(71) Applicant: Freie Universitaet Berlin, Berlin (DE)

(72) Inventors: Volker A. Erdmann, Berlin (DE); Eliza Wyszko, Poznan (DE)

(21) Appl. No.: 14/486,870

(22) Filed: Sep. 15, 2014

Related U.S. Application Data
(63) Continuation of application No. 13/852,111, filed on Mar. 28, 2013, now abandoned.
Application No. 13/852,111 is a continuation of application No. 13/148,142, filed on Aug. 5, 2011, now abandoned.

(30) Foreign Application Priority Data
Feb. 6, 2009 (DE) 10 2009 007 929.7
Aug. 12, 2009 (DE) 10 2009 036 965.1

(51) Int. Cl. A61K 47/48; A61K 31/7088

(52) U.S. Cl. CPC A61K 47/484; A61K 31/7088
USPC 424/178.1; 514/44 A; 536/23.1

(57) ABSTRACT

The invention relates to the use of an L-ribozyme, which is capable of splitting an L-RNA in the region of a target sequence of the L-RNA, in order to produce a pharmaceutical composition for trating undesired physiological adverse reactions due to the administration of a therapeautic modecule containing the L-RNA. Alternatively, an endogeneous target RNA may also be split by the L-ribozyme.

### CROSS REFERENCE TO RELATED APPLICATION

This application is a continuation of U.S. application Ser. No. 13/852,111, filed Mar. 28, 2013, entitled “Pharmaceutical Composition For Treating Adverse Reactions Due To Administration Of Spiegelmers”, which is a continuation of U.S. patent application Ser. No. 13/148,142, filed Aug. 5, 2011, entitled “Pharmaceutical Composition For Treating Adverse Reactions Due To Administration Of Spiegelmers”, which is a 371 national phase filing of PCT/DE2010/000159, filed Feb. 8, 2010, which claims priority to German Application No. 10 2009 007 929.7, filed Feb. 6, 2009 and German Application No. 10 2009 036 965.1, filed Aug. 12, 2009, the disclosures of which are incorporated in their entirety by reference herein.

### TECHNICAL FIELD OF THE INVENTION

The invention relates to the use of an L-ribozyme for producing a pharmaceutical composition, a pharmaceutical composition containing said L-ribozyme and a method for producing said pharmaceutical composition.

### BACKGROUND OF THE INVENTION AND PRIOR ART

Aptamers are generally double-stranded D-nucleic acids, which bind specifically to any target molecule, similarly to an antibody/antigen reaction (Ellington, A. D. et al., Nature 346:818-822 (1990)). Specific aptamers for a given target molecule are isolated for example by the SELEX process from nucleic acid libraries (Tuerk, C. et al., Science 249:505-510 (1990)).

The purpose of aptamers, in the therapeutic range, is among other things to bind and thereby inhibit undesirable metabolic products. In this connection we need only mention for example oncogenic gene products. A disadvantage in the therapeutic use of aptamers is that they have unfavorable pharmacokinetics, i.e. are very rapidly degraded, for example by endogenous nucleases. Independently of this, aptamers are also relatively small molecules, which are therefore excreted relatively quickly via the kidneys.

Spiegelmers are in essence aptamers, but differ from them in that they are formed from L-nucleotides. Spiegelmers can be single-stranded or double-stranded. Through the use of L-nucleotides, degradation by endogenous nucleases is prevented and the pharmacokinetics is thus considerably improved, i.e. the residence time in the serum is prolonged. Thus, in the reference Boisgard, R et al., Eur Journal of Nuclear Medicine and Molecular Imaging 32:470-477 (2005), it is described that nonfunctional Spiegelmers are completely stable metabolically even for a period of 2 hours. The diagnostic use of Spiegelmers is also described in this reference, wherein the Spiegelmer is coupled with a, for example radioactive, reporter substance.

Specific Spiegelmers for a given target molecule can be identified for example as described in the reference Klussmann, S. et al., Nat Biotechnol 14:1112-1115 (1996). Regarding the Spiegelmers and their possible therapeutic applications, reference may also be made to Vater, A. et al., Curr Opin Drug Discov Devel 6:253-261 (2003).

In the therapeutic application of Spiegelmers, up to now it has been assumed that Spiegelmers are not immunogenic (Wlotzka et al., Proc Natl Acad Sci USA 99:8898-8902 (2002)). However, investigations that are described in the present description show that, in an organism, L-nucleic acids are by no means necessarily free from side-effects. Hence it follows that when using Spiegelmers there is certainly a nonnegligible risk of an undesirable physiological side reaction, for example an immune reaction and/or an undesirable enzymatic reaction with endogenous RNA (including a regulatory RNA), on administration to a patient. In particular in the light of the negative experience with the monoclonal antibody TGN1412 in the Phase 1 clinical trial and against the background that the residence time of Spiegelmers, based on the relations mentioned above, is comparatively very high, it would be desirable to have an antidote to a Spiegelmer that is to be used, ready when administering the Spiegelmer, so that if there is an undesirable physiological side reaction the antidote can be administered without delay and the level of Spiegelmer in the serum can be lowered quickly.

From other contexts, namely the ribozyme-catalyzed stereoselective Diels-Alder reaction, L-ribozymes are known, for which reference may be made to Seelig, B. et al., Angew.Chem. Int., 39:4576-4579 (2000) and Seelig, B. et al., Angew. Chem. 112:4764-4768 (2000).

### TECHNICAL PROBLEM OF THE INVENTION

The invention is therefore based on the problem of providing an antidote for Spiegelmers used therapeutically.

### SUMMARY OF THE INVENTION

For solving this technical problem, the invention teaches the use of an L-ribozyme for producing a pharmaceutical composition, wherein the L-ribozyme is able to cleave an L-RNA in the region of a target sequence of the L-RNA, and in particular for producing a pharmaceutical composition for treating undesirable physiological side reactions, in particular immune reactions and/or undesirable enzymatic reactions of the L-RNA with endogenous RNA (including a regulatory RNA), owing to the administration of a therapeutic molecule containing the L-RNA.

The invention is based firstly on the surprising finding that Spiegelmers, contrary to existing assumptions, are not necessarily free of adverse reactions, but rather can be capable of cleaving nucleic acids that occur naturally in an organism and thus producing unforeseeable adverse reactions. The invention is based on this finding, building on the technical teaching of making L-ribozymes available, which specifically cleave a Spiegelmer that has been administered and thus destroy its physiological efficacy, in particular with respect to undesirable side reactions. Examples of Spiegelmers are: Spiegelmer, NOXC89, NOXA42, NOXA50, NOXB11, NOXA12, NOXE36, NOXF37 (all NOXXON AG), Spiegelmers from the company Eli Lilly & Co., NU172 from the company ARCA biopharma Inc., ARCHEMIX, ARC1905, ARC1779, ARC183, ARC184, E10030, NU172, REG2, REG1 (all Archemix Corp.), AS1411, AS140 (both Antisoma Research Ltd.), DsiRNA from Dicerna Pharmaceuticals Inc., RNA Aptamer BEXCORE from BexCore Inc., ELAN from the company Elan Corp Plc, or Macugen. By administering such a ribozyme following the observation of an undesirable side reaction on administration of a Spiegelmer, the cause of the undesirable side reaction can therefore be removed from the metabolism rapidly, effectively and highly selectively, and moreover at extremely low risk of adverse reactions from the administration of the L-ribozyme. The latter is based not only on the construction of the L-ribozyme from L-nucleotides, but additionally on the high selectivity of the L-ribozyme, namely directed onto the target sequence of the Spiegelmer. As a result, a highly effective and highly selective antidote against a therapeutically used Spiegelmer is obtained and undesirable side reactions of the Spiegelmer can be countered effectively, rapidly and without side-effects.

Basically, against any RNA molecule, whether made up of D- or L-nucleotides, it is possible to construct a specific ribozyme, which cuts and thus cleaves a target sequence of the RNA molecule. An essential property of a ribozyme is thus the sequence-specific binding of the ribozyme to the target sequence. However, this also means that for any target sequence, a partial sequence of a ribozyme can be prepared in such a way that the partial sequence of the ribozyme, containing the cleavage site, hybridizes to the target sequence. Therefore, within the scope of the invention, it is not expedient for only particular ribozyme partial sequences to be defined structurally with respect to particular target sequences. The target sequences and ribozyme partial sequences given in the examples are therefore only illustrations and a person skilled in the art can readily determine the appropriate, namely hybridizing ribozyme partial sequence for each given target sequence of a Spiegelmer and synthesize the ribozyme with the usual technical means on the basis of the information on the ribozyme partial sequence.

Basically, the therapeutic molecule can be a Spiegelmer, or the L-RNA can be bound covalently to an aptamer. This last-mentioned case may occur for example in the case of an aptamer stabilized against nucleases. Then the therapeutic benefit of the invention is that by cutting the L-RNA, the aptamer is made accessible for nucleases, so that finally even an aptamer that is causing adverse reactions can be eliminated comparatively quickly from the serum.

However, it is also possible that the L-ribozyme is bound covalently to an aptamer or an antibody. In that case the aptamer or the antibody can for example be selected so that owing to the interactions of the aptamer or of the antibody with cell surfaces, the total construct of L-ribozyme and aptamer or antibody is introduced into the cell.

Preferably the L-ribozyme is a hammerhead ribozyme. Hammerhead ribozymes have a conserved region possibly with a triplet GUH (H is not guanine, preferably C) or a doublet UH (H as above). Regarding the former, reference may be made to FIG. 1. Regarding the latter, reference may be made to Usman, N, et al., The Journal of Clinical Investigation, 106 (10):1197-1201 (2000). Here, the nucleotides N′ and N are any bases, which are selected in the region of the stems I and III according to the target sequence. Essentially, the procedure for constructing an L-ribozyme against a target sequence is first to specify a target sequence, for example a Spiegelmer, wherein said target sequence must contain the triplet GUH or the doublet UH. Then on both ends of a triplet GUH or of the doublet UH, typically in each case 4-10 or 4-11, in particular 6-8 or 6-9, nucleotides are added, whose sequences correspond to the sequences of the target sequence. A copy of the target sequence containing the triplet GUH or the doublet UH is thus obtained, containing 11 to 23 nucleotides. Then the catalytic hammerhead sequence, as shown in FIG. 1, is inserted between the two ends of the copy. An example of a suitable catalytic hammerhead sequence is thus:
Seq-ID 4: 5′-CUGANGAGN′CN′NNNNNGNCGAAAC-3′ or
Seq-ID 5: 5′-CUGANGAGN′CN′NNNNNGNCGAAAN-3′ (N=any bases, wherein in FIG. 1, N and N′ opposite one another necessarily form identical or different base pairs)

This is joined at the 3′-end to nucleotides in the sequence complementary to the target sequence in the 5′-direction of the triplet GUH or doublet UH and at the 5′-end to nucleotides in the sequence corresponding to the target sequence in the 3′-direction of the triplet GUH or doublet UH.

In a preferred embodiment the catalytic hammerhead sequence is Seq-ID 6: 5′-CUGANGAGNUCGGAAACGACGAAAC-3′ or Seq-ID 7: 5′-CUGANGAGNUCGGAAACGACGAAAN-3′ (N=any bases, wherein in FIG. 1, N and N′, which are opposite to one another, necessarily form identical or different base pairs)

Additionally, the sequence 3′-(N)₄₋₆GGUAUAGAGUGCUGAAUCC-5′ (Seq-IDs 8 through 10) can be established at the 5′-end of the catalytic hammerhead sequence, so that a hammerhead ribozyme is obtained, which requires a comparatively low Mg-ion concentration.

The pharmaceutical composition contains the L-ribozyme in at least the dose that corresponds to the dose of administration of the L-RNA, and preferably contains it in a dose that corresponds to 2-10 times the dose of administration of the L-RNA, relative to the moles or number of molecules. An overdosage, compared with the dose of the L-RNA, is recommended, to ensure that all L-RNA to be eliminated is reacted. The absolute doses envisaged according to the invention are based strictly, in the stated relative proportions, on the specified doses of the L-RNA and can therefore easily be determined and established by a person skilled in the art, knowing the specified doses for the L-RNA.

In a preferred embodiment of the invention, the pharmaceutical composition additionally contains a nucleic acid, in particular a 5- to 20-mer, which is capable of the fusing-on of a double-stranded L-RNA in the region of its target sequence. These are sequences that hybridize to partial sequences that are adjacent to the target sequence. As a result, GUC regions of the L-RNA, which normally are not accessible for steric reasons owing to the tertiary structure of the L-RNA, are made accessible for the L-ribozyme.

The invention further relates to a pharmaceutical composition containing an L-ribozyme for treating undesirable physiological side reactions, in particular immune reactions, due to the administration of a therapeutic molecule containing the L-RNA.

With respect to the pharmaceutical composition, all the above and subsequent details apply similarly.

Finally the invention relates to a method for producing said pharmaceutical composition, wherein a sequence is prepared and synthesized from L-nucleotides, which is capable of cleaving a given sequence of L-ribonucleotides, in particular containing the triplet GUC with otherwise any sequences attached upstream and downstream of the triplet, and wherein the L-ribozyme is intended for administration in a pharmacologically effective dose. Typically, the L-ribozyme is mixed with pharmaceutical excipients and/or carriers.

Basically one or more physiologically compatible excipients and/or carriers can be mixed with the L-ribozyme and the mixture can be designed pharmaceutically for local or systemic administration, in particular oral, parenteral, for infusing into a target organ, for injection (e.g. i.v., i.m., intracapsular or intralumbar), for application in tooth pockets (space between tooth root and gum) and/or for inhalation. The choice of additives and/or excipients will depend on the selected dosage form. The pharmaceutical preparation of the pharmaceutical composition according to the invention can take place in the usual manner. As counterions for ionic compounds, for example Mg⁺⁺, Mn⁺⁺, Ca⁺⁺, CaCl⁺, Na⁺, K⁺, Li⁺ or cyclohexylammonium, or Cl⁻, Br⁻, acetate, trifluoroacetate, propionate, lactate, oxalate, malonate, maleate, citrate, benzoate, salicylate, putrescine, cadaverine, spermidine, spermine, etc. may be considered. Suitable solid or liquid pharmaceutical dosage forms are for example granules, powder, coated tablets, tablets, (micro-) capsules, suppositories, syrups, juices, suspensions, emulsions, drops or solutions for injection (i.v., i.p., i.m., s.c.) or nebulization (aerosols), dosage forms for dry powder inhalation, transdermal systems, and preparations with sustained release of active substance, for production of which usual excipients find application, such as carriers, disintegrants, binders, coating materials, swelling agents, glidants or lubricants, tastants, sweeteners and solubilizers. It is also possible to encapsulate the active substance in preferably biodegradable nanocapsules, for example for making a preparation for inhalation. As excipients, we may mention for example magnesium carbonate, titanium dioxide, lactose, mannitol and other sugars, talc, lactoprotein, gelatin, starch, cellulose and derivatives thereof, animal and vegetable oils such as cod-liver oil, sunflower, peanut or sesame oil, polyethylene glycols and solvents, such as sterile water and monohydric or polyhydric alcohols, for example glycerol. A pharmaceutical composition according to the invention can be produced by mixing at least one substance combination used according to the invention in a defined dose with a pharmaceutically suitable and physiologically compatible carrier and optionally further suitable active substances, additives or excipients with a defined dose and processing to the desired dosage form. Polyglycols, water and buffer solutions may be considered as diluents. Suitable buffer substances are for example N,N′-dibenzylethylenediamine, diethanolamine, ethylenediamine, N-methylglucamine, N-benzylphenethylamine, diethylamine, phosphate, sodium bicarbonate, or sodium carbonate. However, it is also possible to work without diluent. Physiologically compatible salts are salts with inorganic or organic acids, for example lactic acid, hydrochloric acid, sulfuric acid, acetic acid, citric acid, p-toluenesulfonic acid, or with inorganic or organic bases, for example NaOH, KOH, Mg(OH)₂, diethanolamine, ethylenediamine, or with amino acids, such as arginine, lysine, glutamic acid etc. or with inorganic salts, such as CaCl₂, NaCl or free ions thereof, such as Ca²⁺, Na⁺, Cl⁻, SO₄²⁻ or corresponding salts and free ions of Mg⁺⁺ or Mn⁺⁺, or combinations thereof. They are produced according to standard methods. Preferably a pH is established between 5 and 9, especially between 6 and 8.

A variant of the invention, which comprises the use of an L-ribozyme for producing a pharmaceutical composition for treating or preventing diseases that are associated with overexpression of at least one endogenous gene, wherein the L-ribozyme is capable of cleaving a target sequence of an endogenous target D-RNA coding for the gene, is important in its own right. Otherwise the above statements apply similarly. In this connection, in another important variant of the above aspect of the invention an L-ribozyme is used for producing a pharmaceutical composition for treating or preventing diseases that are associated with infection of a mammal with a microorganism, wherein the L-ribozyme is capable of cleaving a target sequence of a target D-RNA coding for a gene of the microorganism. Viruses, bacteria and fungi, among others, may be mentioned as microorganisms that may be considered. Basically the ribozyme can be used for the cleavage of any microorganism with at least partially known gene sequences, wherein regions of the gene sequences are selected for the purpose of cleavage, which for example attenuate or inhibit the activity of the microorganism and/or its capacity for replication and/or attenuate or inhibit binding to cell surfaces.

This variant makes use of the fact that L-ribozymes can also be used for cleaving D-RNA, in particular mRNA or regulatory RNA, for example, but not exclusively, siRNA, microRNA, shRNA, ncRNA, tRNA, rRNA, etc. In this way genes or proteins encoded by them can be inhibited. This is of therapeutic benefit for all diseases that are associated with the overexpression of particular genes, compared with the expression in the non-diseased organism.

This variant has on the one hand the advantage that cleavage of the target sequence takes place with very high specificity and therefore there is also no other interference with the regulatory system. Moreover, adverse reactions, such as are associated for example with the use of inhibitory D-nucleic acids, such as siRNA, are reliably avoided.

### BRIEF DESCRIPTION OF THE DRAWINGS

The invention is explained in more detail below, on the basis of figures and examples. The figures show:

FIG. 1A shows a minimal hammerhead ribozyme (Seq-ID 4) before binding to a target sequence (Seq-ID 11) and FIG. 1B shows a hammerhead head ribozyme after binding to a target sequence (Seq-ID 11).

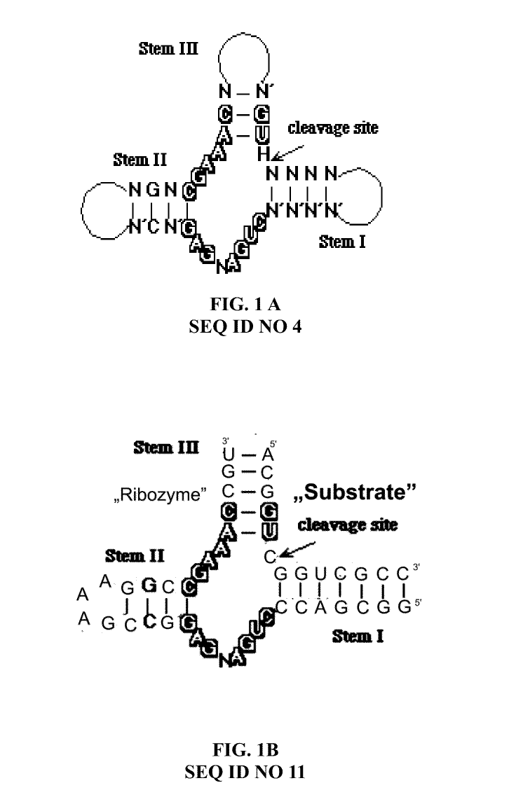

FIG. 2A shows an analysis of the reaction of D-target with L-ribozyme as a function of the MgCl2 concentration, FIG. 2B shows an analysis of the treatment of L-target with D-ribozyme as a function of the MgCl2 concentration and FIG. 2C shows that the proportion of cleavage products of the D-target by an L-ribozyme increases with increasing Mg concentration.

FIG. 3A shows an analysis of the time dependence of the reaction of D-target with L-ribozyme at 10 mM MgCl2 and FIG. 3B shows an analysis of the time dependence of the reaction of L-target with D-ribozyme at 10 mM MgCl2, and FIG. 3C shows that the proportion of cleavage products of the D-target by an L-ribozyme increases with time and is always significantly above the proportion of cleavage products of the L-target.

FIG. 4A shows an analysis of the dependence on MgCl₂ concentration (1-25 mM) of the reaction of L-target with L-ribozyme on the one hand and of D-target with D-ribozyme on the other hand at 10-fold D and L-ribozyme excess and FIG. 4B shows that the proportion of cleavage products of D-target with D-ribozyme and L-target with L-ribozyme increases with increasing Mg concentration.

FIG. 5A shows an analysis of the dependence on MgCl₂ concentration (0.1-1 mM) of the reaction of L-target with L-ribozyme on the one hand and of D-target with D-ribozyme on the other hand at 10-fold L-ribozyme excess and FIG. 5B shows that the proportion of cleavage products of D-target with D-ribozyme and L-target with L-ribozyme over increasing Mg concentration.

FIG. 6A shows an analysis of the time dependence of the reaction of L-target with L-ribozyme at 10 mM MgCl₂ and at 10-fold L-ribozyme excess; FIG. 6B shows shows an analysis of the time dependence of the reaction of D-target with D-ribozyme at 10 mM MgCl₂ and at 10-fold L-ribozyme excess and FIG. 6C shows that the proportion of cleavage products of D-target with D-ribozyme and L-target with L-ribozyme over time.

FIG. 7A shows an analysis of the time dependence of the reaction of L-target with L-ribozyme at 0.1 mM MgCl₂ and at 10-fold L-ribozyme excess, FIG. 7B shows an analysis of the time dependence of the reaction of D-target with D-ribozyme at 0.1 mM MgCl₂ and at 10-fold L-ribozyme excess and FIG. 7B shows that the proportion of cleavage products of D-target with D-ribozyme and L-target with L-ribozyme over time.

FIG. 8A shows an analysis of the time dependence of the reaction of L-target with L-ribozyme at 1 mM MgCl₂ and at 1-fold L-ribozyme excess, FIG. 8B shows an analysis of the time dependence of the reaction of D-target with D-ribozyme 1 mM MgCl₂ and at 1-fold L-ribozyme excess, and FIG. 8C shows that the proportion of cleavage products of D-target with D-ribozyme and L-target with L-ribozyme over time.

FIG. 9A shows an analysis of the time dependence of the reaction of L-target with L-ribozyme at 0.1 mM MgCl₂ and at 10-fold L-ribozyme deficit, FIG. 9B shows an analysis of the time dependence of the reaction of D-target with D-ribozyme at 0.1 mM MgCl₂ and at 10-fold L-ribozyme deficit, and FIG. 9C shows that the proportion of cleavage products of D-target with D-ribozyme and L-target with L-ribozyme over time.

FIG. 10A shows an analysis of the time dependence of the reaction of L-target with L-ribozyme at 1 mM MgCl₂ and at 10-fold L-ribozyme deficit, FIG. 10B shows an analysis of the time dependence of the reaction of D-target with D-ribozyme at 1 mM MgCl₂ and at 10-fold D-ribozyme deficit, and FIG. 10C shows that the proportion of cleavage products of D-target with D-ribozyme and L-target with L-ribozyme over time.

FIG. 11A shows an analysis of the time dependence of the reaction of L-target with L-ribozyme at 5 mM MgCl₂ and at 10-fold L-ribozyme deficit, FIG. 11B shows an analysis of the time dependence of the reaction of D-target with D-ribozyme at 5 mM MgCl₂ and at 10-fold D-ribozyme deficit, and FIG. 11C shows that the proportion of cleavage products of D-target with D-ribozyme and L-target with L-ribozyme over time.

FIG. 12A shows tests on cleavage of L-target by L-ribozyme in human serum and FIG. 12B shows a comparison of the proportion of cleavage products of L-RNA Target versus L-RNA cleavage products in human serum.

### EXAMPLES

### Example 1

### Cleavage Assay

The activities of L-ribozymes and D-ribozymes were measured in various conditions. The basic conditions were as follows. 0.02 μM target RNA was incubated with 10 μl reaction mixture in the presence of 0.002 μM, 0.02 μM and 2 μM ribozyme in 50 mM Tris-HCl buffer, pH 7.5, at 20° C. for 2 hours (ribozymes/target ratio therefore 10:1, 1:1 and 1:10). Before the reaction, target RNA and ribozyme were denatured for 2 minutes at 70° C. and cooled slowly (1° C./min) in the heating unit to 25° C. The influence of the Mg²⁺ ions at concentration from 0.1 to 25 mM was investigated. Cleavage products were separated on 20% polyacrylamide gel electrophoresis in the presence of 8 M urea in 0.09 M Tris-borate buffer, pH 8.3. The fluorescence was analyzed on Phosphoimager Fuji Film FLA 5100. The data were obtained with the program Fuji Analysis Program. Diagrams were prepared with Excel.

### Example 2

### Preparation of the Target Sequences and Ribozymes

The following were prepared as target sequences by way of contract synthesis by the company ChemGenes Corporation, Wilmington, USA:
Seq-ID 1: 5′-FAM-ACAGUCGGUCGCC-3′ (RNA, both with D-nucleotides and with L-nucleotides) and
Seq-ID 2: 5′-FAM-ACAGTCGGTCGCC-3′ (DNA, both with D-nucleotides and with L-nucleotides). The synthesis products had a purity of over 90%.

As ribozyme sequences, depending on the target sequences, the variable regions of a hammerhead ribozyme were selected by the triplet GUC and the following ribozyme sequences were prepared by the company ChemGenes Corporation, Wilmington, USA:
Seq-ID 3: 5′-FAM-GGCGACCCUGAUGAGGCCGAAAGGCCGAAACUGU-3′ (RNA, both with D-nucleotides and with L-nucleotides) The synthesis products had a purity of over 85%. All synthesis products were labeled with fluorescein at the 5′-end.

### Example 3

### Interactions of L-Nucleic Acids with D-Nucleic Acids

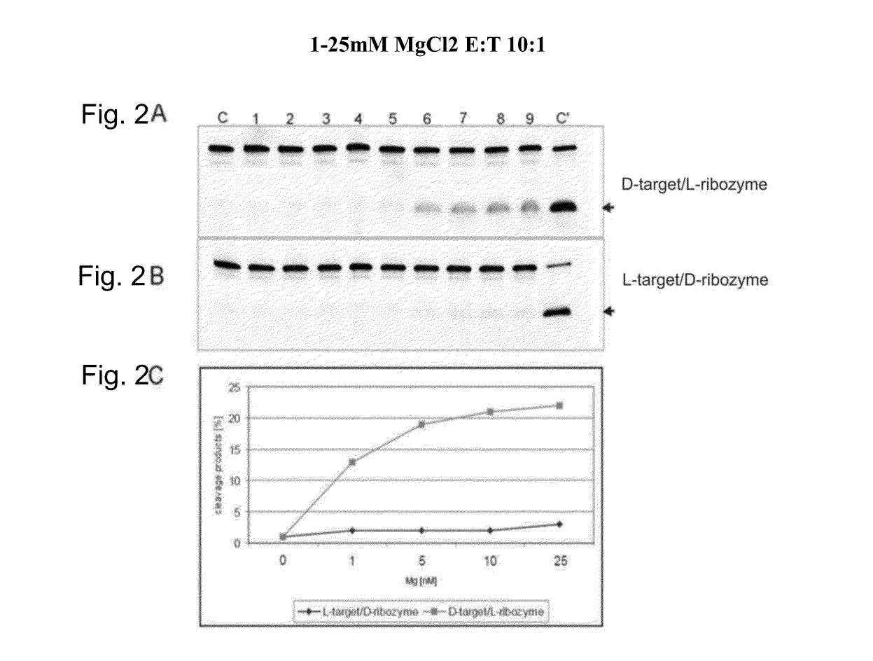

FIG. 2 shows the concentration dependence of the cleavage of a D-target by an L-ribozyme and vice versa. C is the control (L-target+L-ribozyme), tracks 1 to 5 are the various MgCl₂ concentrations given in the diagram (0-25 mM) for target without ribozyme, tracks 6 to 9 0.2 μM target with 2 μM ribozyme.

It can be seen that D-ribozyme does not cleave L-target, but conversely a notable reaction certainly occurs. This means that for example Spiegelmers, consisting of L-nucleotides, in addition to their action as specific aptamer for a given 3-D structure, contrary to the existing notion might certainly be able to engage in further physiological interactions, for example as ribozyme.

Hence it follows that Spiegelmers pose the risk of an undesirable side-effect on administration to an organism.

However, it also follows that L-ribozymes can be used for the cleavage of endogenous D-RNA, leading to therapeutically desired inhibition of the gene or protein coded by the D-RNA, for example mRNA.

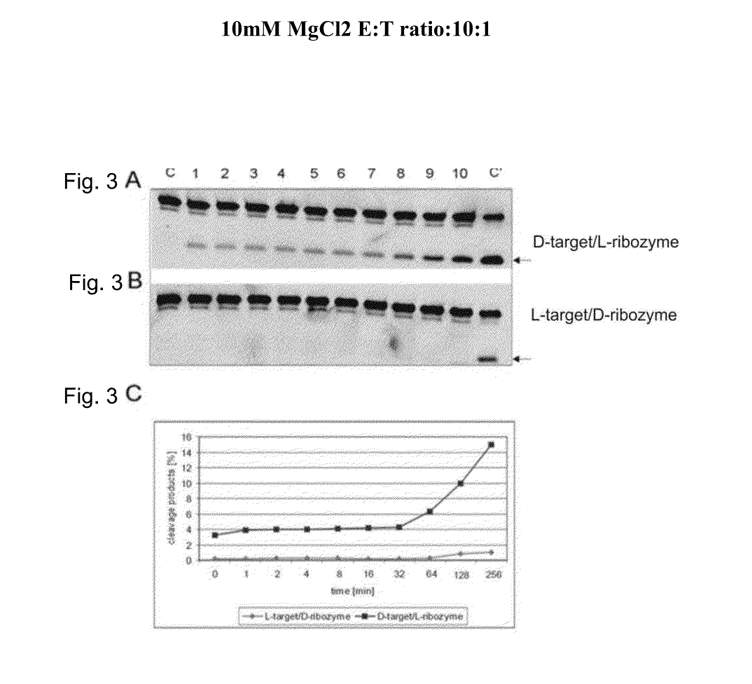

FIG. 3 shows that the proportion of cleavage products of the D-target by an L-ribozyme increases with time and is always significantly above the proportion of cleavage products of the L-target (track C: control, as above, tracks 1 to 10, times 0 to 256 min of the diagram).

### Example 4

### Cleavage of an L-Target by L-Ribozymes

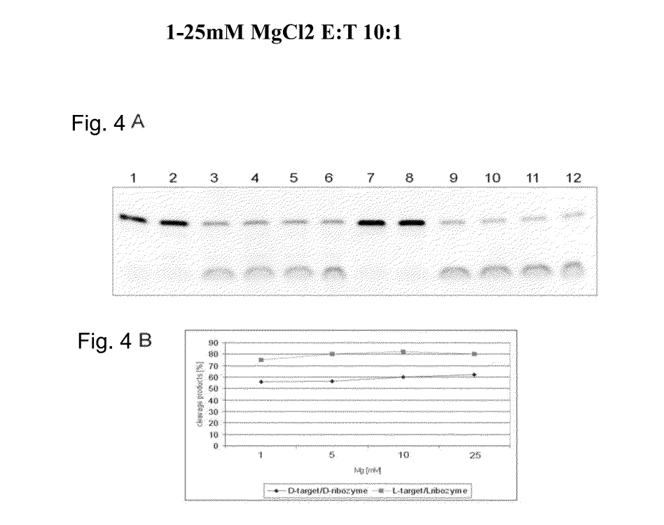

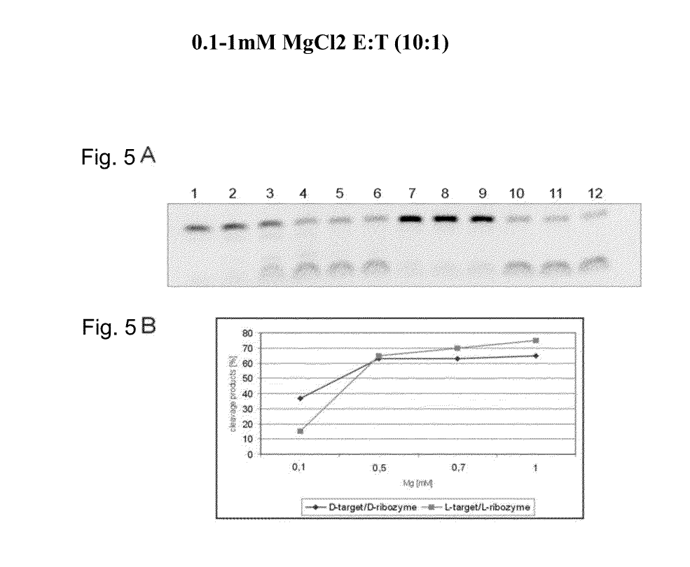

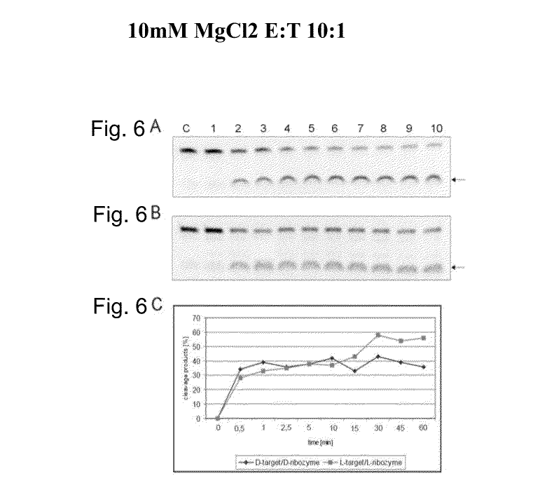

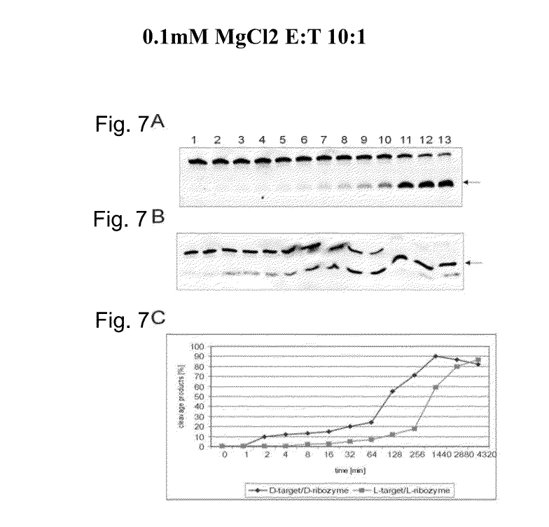

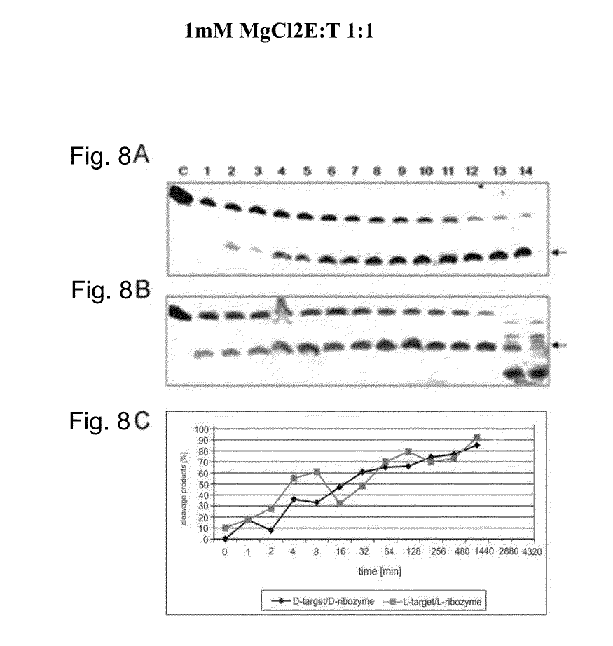

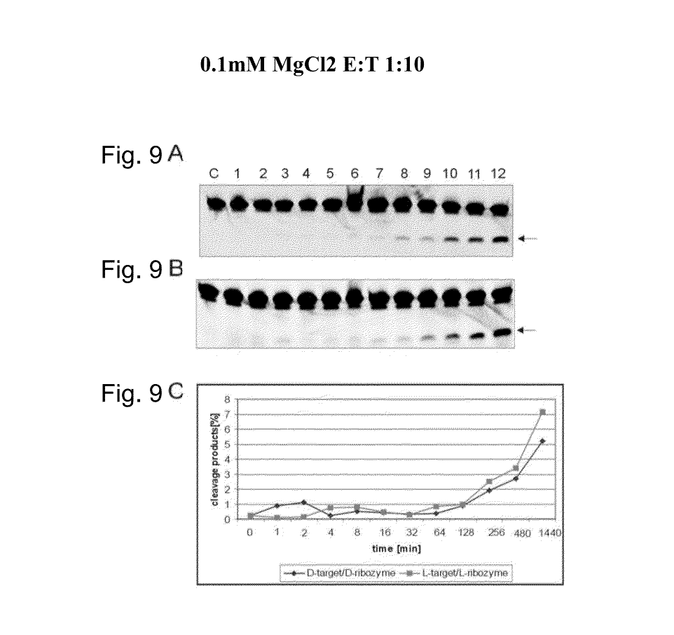

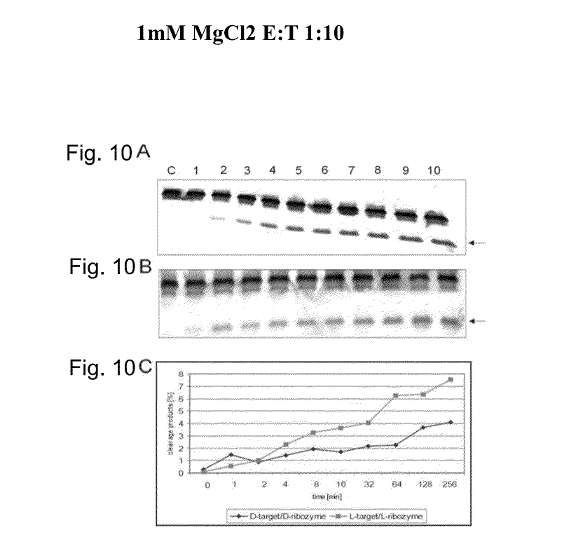

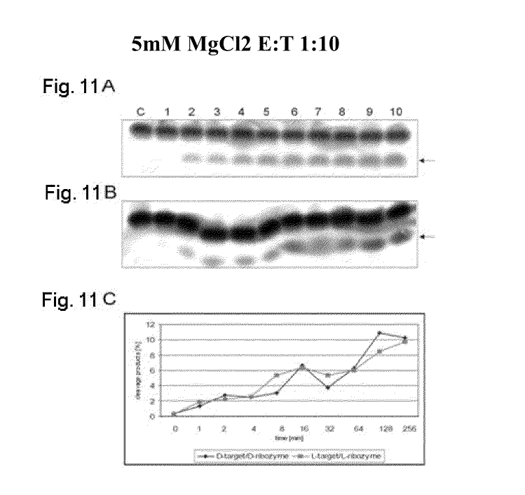

It can be seen from FIGS. 4 to 11 that an L-ribozyme effectively cuts an L-target with corresponding target sequence in all usual conditions, and moreover with turnover rates that at least correspond to those of a D-ribozyme with a D-target.

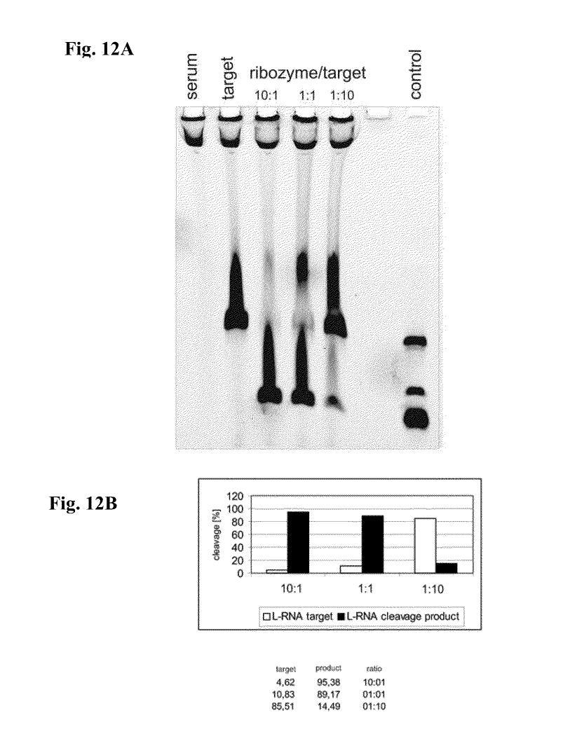

FIG. 12 provides evidence that the cleavage of an L-target by an L-ribozyme also functions effectively under the conditions of human serum.

1. A method of treatment or prevention of a medical condition comprising administering a patient in need thereof with a pharmaceutical composition comprising an L-ribozyme.

2. The method of claim 1, wherein the L-ribozyme is capable of cleaving an L-RNA in the region of a target sequence of the L-RNA.

3. A method of treatment of undesirable physiological side reaction due to administration of a therapeutic molecule containing a L-RNA comprising administering a patient in need thereof with a pharmaceutical composition comprising an L-ribozyme, which is capable of cleaving the L-RNA in the region of a target sequence of the L-RNA.

4. A method of treatment or prevention of diseases which are associated with overexpression of at least one endogenous gene comprising administering a patient in need thereof with a pharmaceutical composition comprising an L-ribozyme, wherein the L-ribozyme is capable of cleaving a target sequence of an endogenous target D-RNA coding for the gene.

5. The method as claimed in claim 3, wherein the therapeutic molecule consists of the L-RNA, in particular is a double-stranded L-RNA, for example a Spiegelmer.

6. The method as claimed in claim 3, wherein the therapeutic molecule contains an aptamer bound covalently to the L-RNA or antibody bound covalently thereto.

7. The method as claimed in claim 3, wherein the pharmaceutical composition comprises the L-ribozyme in at least the dose corresponding to the dose of administration of the L-RNA, preferably comprises it in a dose that corresponds to 2 to 100 times, preferably 2 to 20 times the dose of administration of the L-RNA.

8. The method as claimed in claim 3, wherein the L-ribozyme is a hammerhead ribozyme.

9. The method as claimed in claim 3, wherein the pharmaceutical composition additionally comprises a nucleic acid, in particular a 5- to 20-mer, which is capable of the fusing-on of a double-stranded D-RNA or L-RNA in the region of the target sequence.

10. A pharmaceutical composition comprising an L-ribozyme with the capability of cleaving an L-RNA in the region of a target sequence of the L-RNA for reducing undesirable physiological side reactions, due to the administration of a therapeutic molecule containing the L-RNA.

11. A pharmaceutical composition containing an L-ribozyme for preventing diseases which are associated with overexpression of at least one endogenous gene, wherein the L-ribozyme is capable of cleaving a target sequence of an endogenous target D-RNA coding for the gene.

12. A method of making of a pharmaceutical composition of claim 10 or claim 11 comprising the steps of preparing and synthesizing a sequence of L-nucleotides, which is capable of cleaving a given sequence of L-ribonucleotides or a given sequence of D-ribonucleotides, and preparing the L-ribozyme for administration in a pharmacologically effective dose.

13. The method of claim 12, wherein the L-ribozyme is mixed with pharmaceutical excipients and/or carriers.

14. The method of claim 3, wherein the dose of administration corresponds to 2 to 100 times the dose of administration of the L-RNA.

15. The method of claim 3, wherein the dose of administration corresponds to 2 to 20 times the dose of administration of the L-RNA.
